(19) United States
(12) Patent Application Publication
(10) Pub. No.: US 2010/0198225 A1
(43) Pub. Date: Aug. 5, 2010

(54) SHAPE MEMORY SPINE JACK

(76) Inventors: Andrew ...Nmi Thompson, York (GB); Graeme Nmi Howling, Leeds (GB); Alan Nmi Horner, Selby (GB)

Correspondence Address: DIANA HOUSTON;SMITH & NEPHEW, INC., 1450 BROOKS ROAD, MEMPHIS, TN, 38116, US

(21) Appl. No.: 12/596,147

(22) PCT Filed: Apr. 15, 2008
(86) PCT No.: PCT/GB08/01314

§ 371 Date: Apr. 20, 2010

(30) Foreign Application Priority Data
Apr. 17, 2007 (GB) 0707394.3

(51) Int. Cl. A61B 17/60

(52) U.S. Cl. 606/90

(57) ABSTRACT

The present invention relates to a method of repairing vertebral bodies and of controlling vertebral height augmentation. In particular, the invention relates to a spine jack and more particularly to a spine jack comprising shape memory material for use in the method of repair.

The present invention relates to a method of repairing vertebral bodies and of controlling vertebral height restoration and augmentation. In particular, the invention relates to a spine jack and more particularly to a spine jack comprising shape memory material for use in the method of repair.

Vertebral compression fractures are a common feature of osteoporosis and can cause a considerable amount of pain and suffering resulting in a significantly reduced quality of life for the patient. Vertbroplasty is a general term used to describe medical procedures whereby a fractured vertebra is stabilised. Typically, vertbroplasty is used to repair fractures caused by osteoporosis. Osteoporotic fractures can lead to a decrease in vertebrae height leading to spinal deformities including hunchbacking. Vertebroplasty reduces pain by stabilising the vertebrae and is commonly performed by a surgeon or a radiologist. Known vertbroplasty procedures involve the percutaneous injection of cement into the fractured site, or the pre-insertion of a balloon followed by cement injection (commonly known as kyphoplasty).

Cements used in such procedures generally include methylmethylacylates which are toxic. A further problem with using the procedure of injecting cement alone is that the cement may leak from the fractured site into sensitive areas such as the spinal column. Such procedures also require the cement to be injected at high pressure which in effect provides the force to raise the height of the vertebra. However, vertbroplasty procedures of the prior art are incapable of guiding this force along the plane required to raise the height of the vertebra but rather deliver the forced in a multitude of directions which in turn exerts force on more sensitive parts of the spinal column which may result in adverse affects and ultimately damage the spine. Furthermore, as the force of the injected cement is misdirected, the cement must be injected under a higher pressure than is actually required to raise the vertebra, which in turn may lead to greater leakages of cement and increase the chances of damaging the spine.

Problems associated with the use of cement alone are addressed to some extent by the kyphoplasty procedure. The procedure of kyphoplasty aims to restore the height and angle of fractured vertebrae through mechanical or hydraulic intervertebral expansion. This is generally achieved using inflated kyphoplasty balloons which distribute load across vertebral endplates within a region of weak bone rather than distributing across the endplate as with other known vertbroplasty procedures. After the balloon has been inserted into the prepared fractured site, it is inflated which provides sufficient force to raise the height of the vertebra. Cement is then passed into the inflated balloon through a hollow needle (trocar). The inflated balloon takes the weight of the vertebra so that the cement does not need to be injected with the high pressure required in the case where cement alone is used. Using balloons to restore the height and angle of fractured vertebrae helps minimise the leakage of cement into sensitive areas such as the spinal column. This is due in part because the inflated balloon takes the load of the vertebra but also because the cement is now contained within the inflated balloon. The surgeon may also monitor the procedure fluoroscopically to limit or prevent cement from escaping the balloon and entering the spinal canal area. Although the kyphoplasty procedure does offer some improvements compared to other known vertbroplasty procedures, it still doesn't maximise vertebral height restoration and some cement leakage may still be observed.

Vertbroplasty procedures of the prior art have limitations in terms of the control of vertebral height restoration. Applying forces to such areas of the human body as the spine can be potentially harmful if not properly controlled and directed. For vertebral height restoration, a controlled force in a single plane only is required. Procedures of the prior art, including kyphoplasty, invariably exert forces in more than one plane. Such additional forces are not only ineffective in restoring the height of the vertebral body but are also potentially harmful.

Further vertbroplasty devices and procedures of the prior art are described in US2006/0095138 and WO2007/038009. These disclosures, however, also fail to provide controlled adjustment of vertebral height.

It is an object of the present invention to provide a device and method which enables tailored expansion of a vertebral body in a controlled manner and along a single plane.

Therefore, according to a first aspect of the present invention there is provided a device comprising shape memory material for increasing the height of an injured or collapsed vertebral body, capable of restoring it to its original height. The shape changing characteristic of the shape memory material is utilised to distract the spinal fracture gap restoring vertebral height. The degree to which the vertebral body is restored may be controlled by the application of a stimulus to the shape memory material and/or altering the composition of the shape memory material.

Generally, the device is inserted into the cavity of the fracture which may be prepared to receive the device. Alternatively, the device may be adapted to fit the shape of the cavity of the fracture. Typically, the height of the vertebral body is the distance between adjacent vertebral discs.

Preferably, the device comprises at least one support element having shape memory properties and being configured such that the support element exerts a force to the vertebral body along a single support plane.

Generally, the support element comprises shape memory material. The shape memory material can be a shape memory alloy, for example, nitinol. Preferably, the shape memory material is a shape memory polymer. Alternatively, the material comprises a mixture of a shape memory alloy or shape memory alloys, and a shape memory polymer or shape memory polymers. The shape, memory polymer can be resorbable/non-resorbable or a combination of both. Specific shape memory polymers that may be used include polyetheretherketone (PEEK), polymethyl methacrylate (PMMA), polyethyl methacrylate (PEMA), polyacrylate, poly-alpha-hydroxy acids, polycapropactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, polyurethanes, and copolymers or polymer blends thereof.

The general concept of shape memory polymer (SMP) is described below. The concept is to utilise the properties of shape memory polymers to give an advantageous vertebroplasty device and method that is superior in terms of procedure and clinical outcome compared to the prior art. Advantages of the present invention over the prior art include: Use of a non-flowable material (SMP) which avoids contamination of sensitive areas with toxic bone cements; does not need high pressure injection systems, thus avoiding tissue damage and toxic bone cement infiltration; provides controlled vertebral height distraction (surgeon can choose precisely how much the vertebrae can be adjusted); is minimally invasive (small implants can be implanted then can be expanded to the desired configuration); and generally directs the force required to raise the height of the vertebra along a single plane for maximum effective uplift of the vertebra, thus avoiding the application of forces in directions which provide less effective uplift and may ultimately cause spinal damage.

Shape memory polymers (SMPs) are materials that have the ability to “memorize” a “permanent” macroscopic shape, be orientated or manipulated under temperature and/or stress to a temporary or dormant shape, and then be subsequently relaxed to the original or memorized, stress-free condition or shape. Relaxation is usually prompted or encouraged by the application of thermal, electrical, or environmental energy to the manipulated or orientated SMP. This relaxation is associated with elastic deformation energy stored in the SMP during orientation of the SMP. The degree of orientation of the SMP is the driving force that causes relaxation. Thus the greater the degree of orientation, the greater will be the force or energy stored in the SMP and hence the greater will be the force or energy driving relaxation of the SMP when triggered or prompted by an external energy source.

SMPs like other polymers can be grouped into two main categories; they can be amorphous, thus lacking any regular positional order on the molecular scale, or they can be semicrystalline which contain both molecularly ordered crystalline regions and amorphous regions in the same sample.

Plastic deformation of amorphous SMPs and SMP composites results in the formation of an orientated amorphous or semi-crystalline polymer network. Orientation of SMPs and SMP composites can be achieved by stretching, drawing or applying a compressive and/or shear force to the SMP. The SMP may be orientated by application of any one or a combination of these forces and can be carried out at ambient temperatures or elevated temperatures. Generally, the temperature of the SMP is raised above ambient temperature to around the glass transition temperature (Tg) of the SMP before application of the orientation force or forces. Raising the temperature of the SMP in this way helps prevent the SMP from rupturing when the orientation force is being applied thereto. The glass transition temperature is the temperature below which the physical properties of amorphous SMPs behave in a manner similar to a solid, and above which they behave more like a rubber or liquid allowing the SMP to undergo plastic deformation without risk of fracture. The glass transition temperature of the SMP will vary based on a variety of factors, such as molecular weight, composition and structure of the polymer, and other factors known to one of ordinary skill in the art, but is generally in the region of between 35-150° C. After the SMP has been orientated, the temperature is reduced and the SMP is fixed in a temporary or dormant configuration.

The orientated network is physically stable well below the glass transition temperature (Tg) where molecular mobility is low. However, near or above the polymer's glass transition temperature, molecular motion rapidly increases and causes the orientated network to relax, usually accompanied by physical changes in the dimensions of the SMP. During relaxation, the orientated SMP tends to recover the original dimensions of the unorientated SMP, hence the name shape “memory” material. However, recovery of the original shape depends primarily on the degree of crystallinity, orientation, the micro and nano-structures and the conditions under which the orientated network is relaxed. For copolymers other important factors are their detailed composition and their specific thermal properties, i.e. the glass transition and melting temperatures, of their components.

It is believed that the relaxation process occurs nearly at constant volume. The degree of recovery during relaxation, for a semi-crystalline orientated SMP, depends on its crystallinity and structure and complete recovery of its original shape is difficult. In contrast, amorphous orientated SMPs, copolymers and their composites can return substantially to their original shape under appropriate relaxation conditions.

The degree of orientation is the driving force that causes relaxation. The greater the degree of orientation, i.e. the force or forces applied to the SMP, the greater will be the driving force.

During relaxation, the orientated SMP releases stored internal forces or energy. For example, an SMP of cylindrical shape orientated by applying a stretching force uniaxially along its longitudinal axis will shrink in length and expand in diameter during relaxation under free boundary conditions, i.e. where no physical constraints are imposed. Hence, when the cylindrical shaped SMP relaxes, it will induce a shrinkage force along its longitudinal axis and also an expanding force in the radial direction. These longitudinal and radial forces are proportional to the degree of orientation and mass of orientated polymer. The greater the degree of orientation, i.e. the greater the forces applied to the SMP during orientation, and the greater the mass of the SMP, the greater these longitudinal and radial relaxation forces will be. For SMPs of other geometries, the relaxation forces will also depend on the degree or magnitude of the orientation force, the direction of the applied orientation force, as well as the mass of the orientated SMP. The rate of relaxation or the rate of shape recovery of the SMP is dependent on sample geometry, processing conditions and more importantly on, the mass and thermal diffusivity of the SMP.

Preferably, the support element comprises an orientated shape memory polymer. However, it will be appreciated that the support element can be pre-stressed or orientated at any time prior to use or insertion into a spinal cavity.

The device is ideally suited as a spine jack as force directed other than along the single support plane required to lift the vertebral body may damage the spinal column. Ideally, said single support plane is parallel to the longitudinal axis of the spinal cord. More specifically, the single support plane is substantially perpendicular to the generally flat load bearing surface of the adjacent discs of the vertebral body being treated.

Forces exerted by the support element to the vertebral body, other than along said single plane are practicably negligible. In cases where the support element exerts forces other than along said single plane, these forces are absorbed by the body of the spine jack and not relayed to the vertebral body.

Preferably, the spine jack includes a buffer region which absorbs the forces exerted by the support element in planes other than in the single support plane. Preferably, the buffer region surrounds at least a portion of the support element. For example, the buffer region can be empty space between the support element and a cavity wall of the fracture. When the support element is prompted to relax, it may expand in more than one plane. However, the buffer region only allows forces from the relaxing support element to be transferred to the spine along the single support plane.

Alternatively, the buffer region can be a low density, deformable medium or matrix such as a, foam, for example. Preferably, the density of the buffer region is between 1.168 kg/m³ and 50 kg/m³ measured at 25° C. and 100 kPa; 1.168 kg/m³ being the approximate value for the density of air. More preferably, the density of the buffer medium is between 1.168 kg/m³ and 10 kg/m³. Preferably, the buffer region is porous.

In use, the foam fills the cavity of the fracture and surrounds the orientated support element. When the support element relaxes it generally expands along one or more planes. Where the support element expands along more than one plane, the foam immediately around the support element absorbs the force of the expanding support element except along said single support plane, preventing the transfer of force to the spine along the additional force planes. The foam immediately around the expanding support element becomes more dense due to the compressive force of the expanding support element. The foam also provides the advantage of stabilising the spine jack within the cavity.

Alternatively, the buffer region restricts or prevents movement or expansion of the support element except along the single support plane. For example, the buffer region may be a solid, non shape changing, component in the form of a plug shaped to fit snugly within the cavity of the fracture. Preferably, the buffer plug includes at least one recess for receiving the at least one support element. Preferably, the at least one recess has at least one opening at an end thereof. Preferably, the support element is orientated to fit snugly within the recess and such that an end of the orientated support element lies flush with said at least one opening. In use, the support element is stimulated to relax. The recess walls of the buffer plug restrict expansion of the support element and directs the entire expansion of the support element through said at least one opening. Preferably, the recess is generally cylindrical in shape to receive the similarly shaped support element and has two openings located at opposite ends of the recess. In this way, expansion of the support element is directed through the openings along the single support plane providing a jacking effect to the collapsed vertebral body.

Preferably, the spine jack includes an interface between the support element and the load bearing surface of the vertebral disc. More preferably, the spine jack includes an interface between the support member and both adjacent vertebral discs. Preferably, said interface is a load bearing plate. In use, the load bearing plate transfers the force from the support member more evenly to the vertebral discs.

Preferably, the support element includes a passageway for receiving an energy probe for relaxing the SMP. Typically, the energy probe is a heating probe. The support element and passageway are advantageously cylindrical in shape and the passageway preferably lies centrally and along a longitudinal axis of the support element. The cylindrical shape of the supporting element is preferable as it provides an easy fit into a fracture which has been prepared using a drill, the drilled hole also being generally cylindrical in shape. Locating the passageway centrally of the supporting element allows the heat from the probe to be more evenly transferred to the SMP of the support element.

Preferably, the supporting element is orientated in a single orientated plane or along a single axis. Preferably, the orientated plane or axis is perpendicular to the single support plane.

Optionally, the spine jack or parts thereof can be porous, semi porous and/or include channels for receiving bone growth enhancing material and/or bone cement. Porosity of the spine jack allows infiltration thereof by cells from surrounding tissues, enhancing integration thereof by osteointegration, for example.

Preferably, the SMP of the support element is biocompatible and can be resorbable or non-resorbable, or a combination of both. Example of suitable SMPs include but are not limited too polyetheretherketone (PEEK), polymethyl methacrylate (PMMA), polyethyl methacrylate (PEMA), polyacrylate, poly-alpha-hydroxy acids, polycapropactones, polydioxanones, polyesters, polyglycolic acid, polyglycols, polylactides, polyorthoesters, polyphosphates, polyoxaesters, polyphosphoesters, polyphosphonates, polysaccharides, polytyrosine carbonates, polyurethanes, and copolymers or polymer blends thereof.

Preferably, the SMP is a reinforced SMP. Preferably, the reinforced SMP comprises a composite or matrix including reinforcing material or phases such as fibers, rods, platelets, and fillers. More preferably, the SMP can include glass fibers, carbon fibers, polymeric fibers, ceramic fibers, or ceramic particulates.

Preferably, the spine jack is coated with an osteogenic material such as, for example, hydroxyapatite or calcium phosphate.

Preferably, one or more active agents are incorporated into the spine jack. Suitable active agents include but are not limited too anti-osteoporotic agents, bisphosphonates, bone morphogenic proteins, antibiotics, anti-inflammatories, angiogenic factors, osteogenic factors, growth factors, monobutyrin, omental extracts, thrombin, modified proteins, platelet rich plasma/solution, platelet poor plasma/solution, bone marrow aspirate, and any cells sourced from flora or fauna, such as living cells, preserved cells, dormant cells, and dead cells.

Preferably, the active agent is incorporated into the spine jack and is released during the relaxation or degradation of the SMP. Advantageously, the incorporation of an active agent can also act to combat infection at the site of implantation and/or to promote new tissue growth.

Typically, the spine jack is used in conjunction with means for delivering the spine jack to the fractured site. Therefore, according to a second aspect of the, present invention there is provided apparatus for increasing the height of a collapsed vertebral body comprising a spine jack as hereinbefore described, a device for delivering the spine jack to the vertebral body and means for stimulating the shape memory material of the support element.

Preferably, the device for delivering the spine jack is a cannula or trocar. Typically, the means for stimulating the shape memory material of the support element is a heating, ultrasound or infrared probe. Said stimulating means may also include alternative devices or ways of transferring energy to the supporting element to promote relaxation of the orientated support element. For example, the temperature of the patient's body fluid may contain sufficient heat energy to promote relaxation of the support element.

Alternatively or additionally, the expansion step may be prompted or triggered by the application of a different form of energy, for example, a magnetic field, an electric current, electromagnetic radiation such as microwaves, visible and infrared light, or by a combination of any one of these forms of energy.

Stimulating molecular motion of the SMP may also be achieved by exposing the orientated SMP to a plasticizer. Exposure of the SMP to a plasticizer reduces the Tg of the SMP, thus increasing its molecular mobility. In this way, the molecular mobility of the orientated SMP may be increased sufficiently to cause the orientated network to relax without the input of energy. Where exposure of the orientated SMP to a plasticizer is not sufficient to relax the SMP, energy, in the form of heat for example, may also be applied to the SMP. In this way, the orientated SMP can be relaxed at a temperature less than would be necessary where the SMP is relaxed using heat alone. As such, the SMP can be shaped at lower temperatures, thus allowing the addition of temperature sensitive materials to the SMP. Temperature sensitive materials may include, for example, releasable bioactive agents such as monobutyrin, bone marrow aspirate, angiogenic and osteogenic factors which will aid bone fracture repair.

Suitable plasticizers may be in the form of a biocompatible volatile liquid or gas. Examples of such gaseous plasticizers include but are not limited to, oxygen and carbon dioxide. Examples of such liquid plasticizers include but are not limited to, water and inorganic aqueous solutions such as sodium chloride solution.

Generally, the present invention contemplates the use of electrical and/or thermal energy sources to transfer energy to the shape memory polymer of the spine jack or to the support element in particular. However, the shape memory polymer can be relaxed via other methods known to those of ordinary skill in the art, including, but not limited to the use of force, or mechanical energy, and/or a solvent. Any suitable force that can be applied either preoperatively or intra-operatively can be used. One example includes the use of ultra sonic devices, which can relax the polymer material with minimal heat generation. Solvents that could be used include organic-based solvents and aqueous-based solvents, including body fluids. Care should be taken that the selected solvent is not contra indicated for the patient, particularly when the solvent is used intra-operatively. The choice of solvents will also be selected based upon the material to be relaxed. Examples of solvents that can be used to relax the shape memory polymer include alcohols, glycols, glycol ethers, oils, fatty acids, acetates, acetylenes, ketones, aromatic hydrocarbon solvents, and chlorinated solvents.

The combination of spine jack, spine jack delivery device and means for stimulating the shape memory material of the support element provides a way of locating the spine jack within the fractured site and raising the height of the collapsed vertebral body. Therefore, according to a third aspect of the present invention there is provided a method for increasing the height of an injured or collapsed vertebral body comprising the steps of introducing the spine jack into the fractured vertebral body and stimulating the support element to promote relaxation of the support element.

Preferably, the fractured vertebral body is surgically prepared before inserting the spine jack therein: Typically, the fractured site is prepared by shaping the site, forming a cavity to receive the spine jack. Shaping of the fractured site can be done with a surgical drill, for example.

Compared with the prior art, the spine jack of the present invention offers numerous advantages including controlled adjustment of vertebral height, a one step procedure and no fluoroscopic guidance is required.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### DETAILED DESCRIPTION OF THE INVENTION

Embodiments of the invention will now be described by way of example only and with reference to the accompanying drawings, in which:

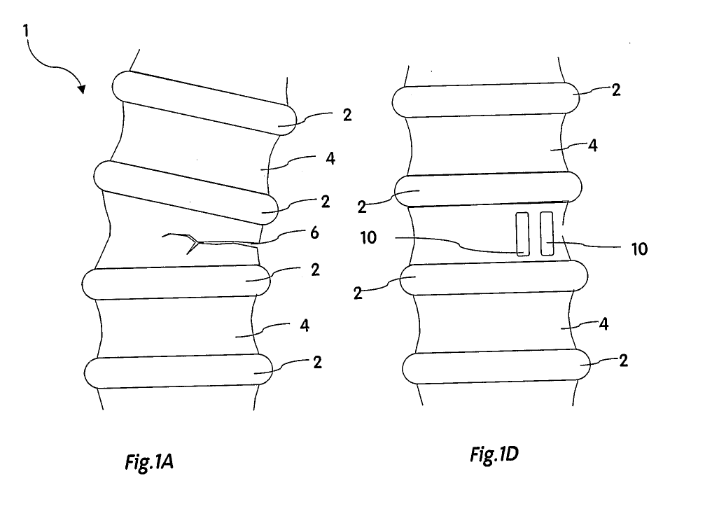

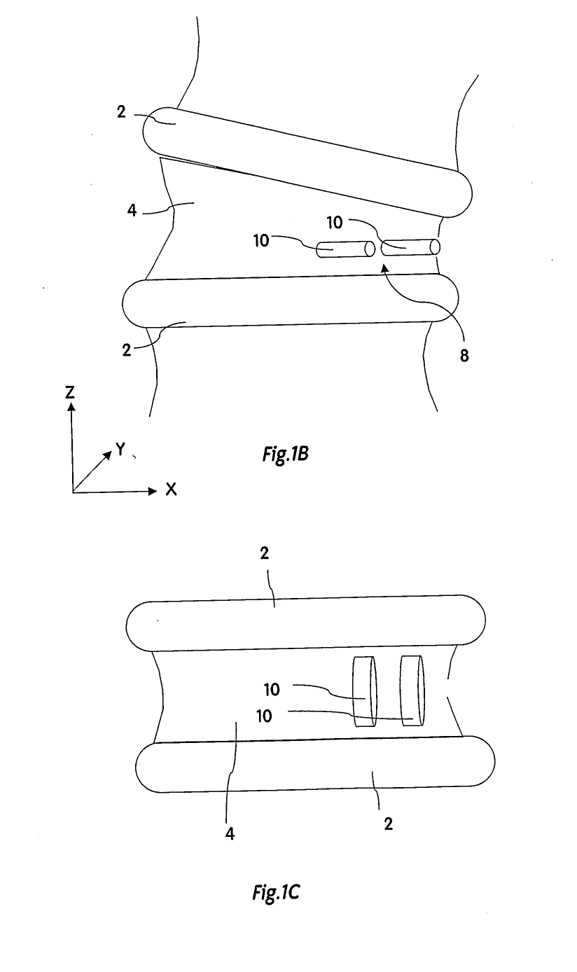

FIG. 1A is a cross sectional side view of a portion of the spine showing intervertebral discs between vertebral bodies, one of the vertebral bodies having a wedge crush fracture;

FIG. 1B is an enlarged cross sectional side view of the spine of FIG. 1A showing a fractured or collapsed vertebral body including the spine jack located within the fracture (fracture cavity not shown), the spine jack having two support elements shown here prior to relaxation;

FIG. 1C is a view of the spine of FIG. 1B, the support elements shown here fully relaxed and the vertebral body repaired and restored to its original height;

FIG. 1D is a similar view of the spine of FIG. 1A after treatment;

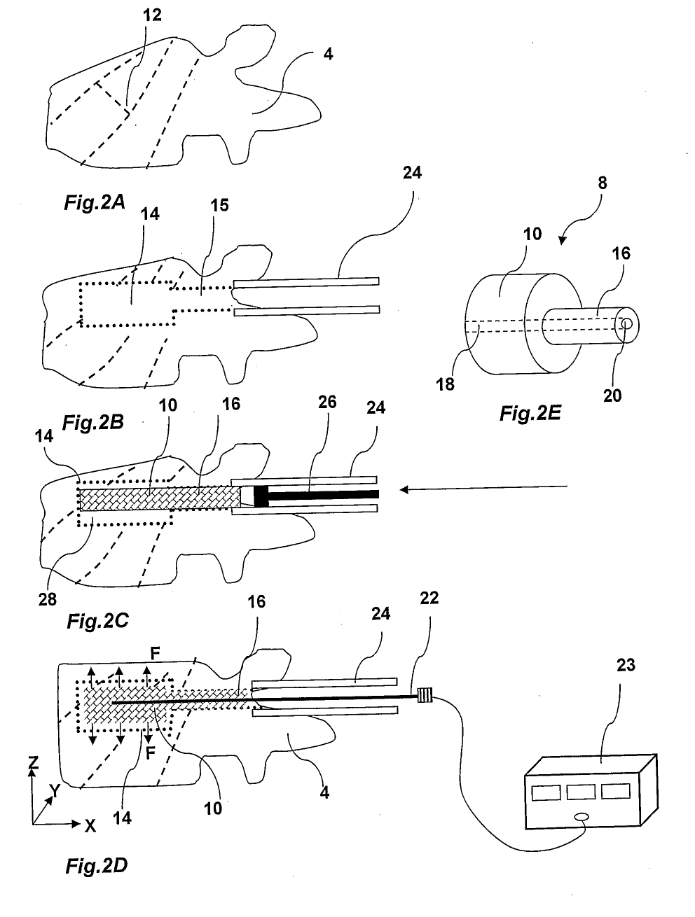

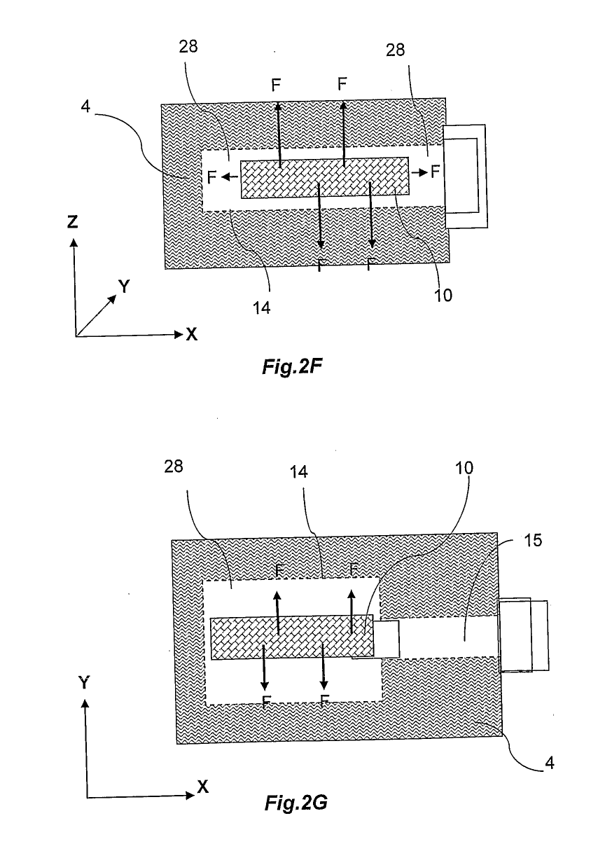

FIG. 2A is a cross sectional side view of a vertebral body illustrated here having compression fractures, the fractures being shown here with hatched or broken lines;

FIGS. 2B-2D illustrates a method of treatment according to the present invention using a spine jack and apparatus of the present invention for restoring the shape and height of the collapsed vertebral body of FIG. 2A;

FIG. 2E illustrates an embodiment of spine jack according to the present invention, the support element shown here as the larger cylindrical member;

FIG. 2F is a cross sectional side view, shown in part, of the spine jack of FIGS. 2B to 2E, showing expansion of the support element in the X and Z planes;

FIG. 2G is a cross sectional plan view, shown in part, of the spine jack of FIGS. 2B to 2E, showing expansion of the support element in the Y plane;

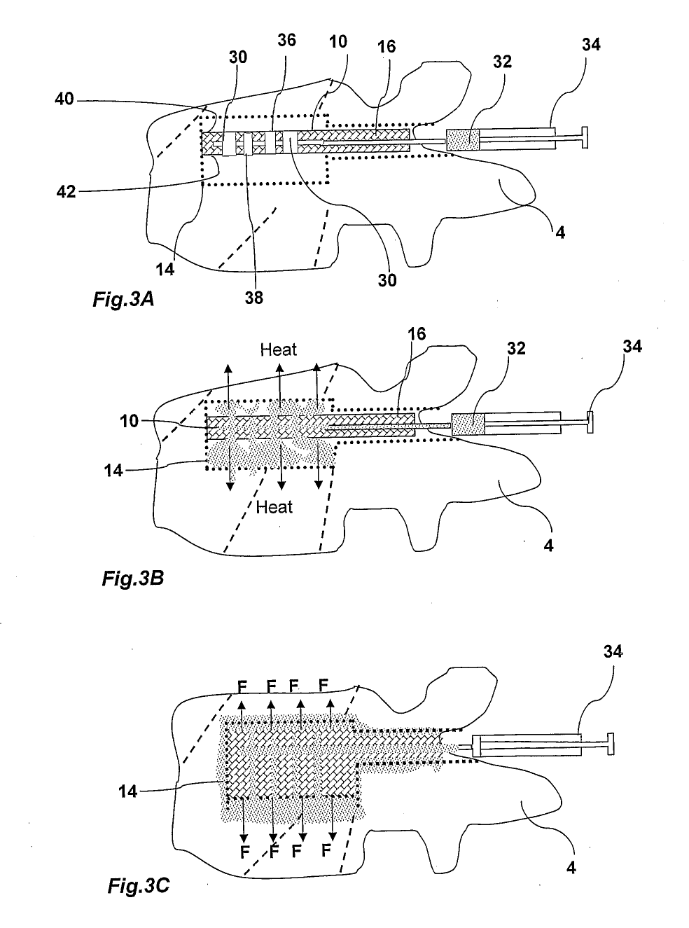

FIG. 3A is a cross sectional side view of a collapsed vertebral body showing an embodiment of spine jack positioned within a prepared cavity in a collapsed vertebral body, the support element shown here in an orientated state and having channels for receiving exothermic agent to relax the shape memory polymer of the support element;

FIG. 3B is a similar view of the collapsed vertebral body of FIG. 3A showing injection of the exothermic agent;

FIG. 3C is a similar view of the vertebral body of FIGS. 3A and 3B showing the support element relaxed by the exothermic agent, the arrows labelled F clearly showing the expansion force of the support element in a single plane repairing and restoring the height of the vertebral body;

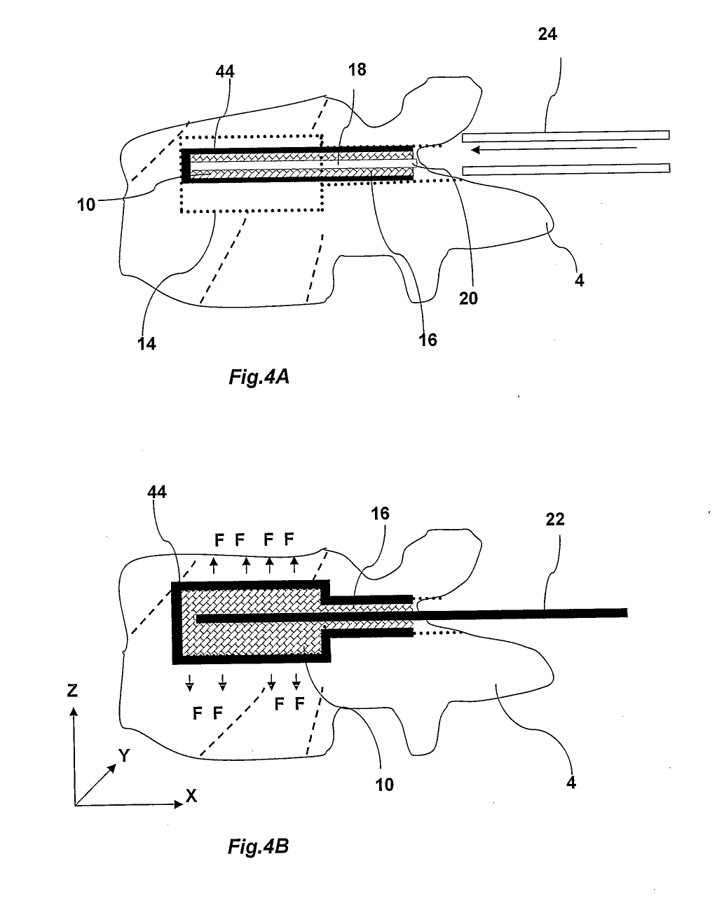

FIG. 4A is a cross sectional side view of a collapsed vertebral body showing an embodiment of spine jack positioned within a prepared cavity in the collapsed vertebral body, the support element shown here in an orientated state and having a central recess for receiving a heating probe to relax the shape memory polymer of the support element, an exterior surface of the support element shown here coated with a material which aids integration with the surrounding bone;

FIG. 4B is a similar view of the vertebral body of FIG. 4A showing the support element relaxed by a heating probe, the arrows labelled F clearly showing the expansion force of the support element in a single plane repairing and restoring the height of the vertebral body;

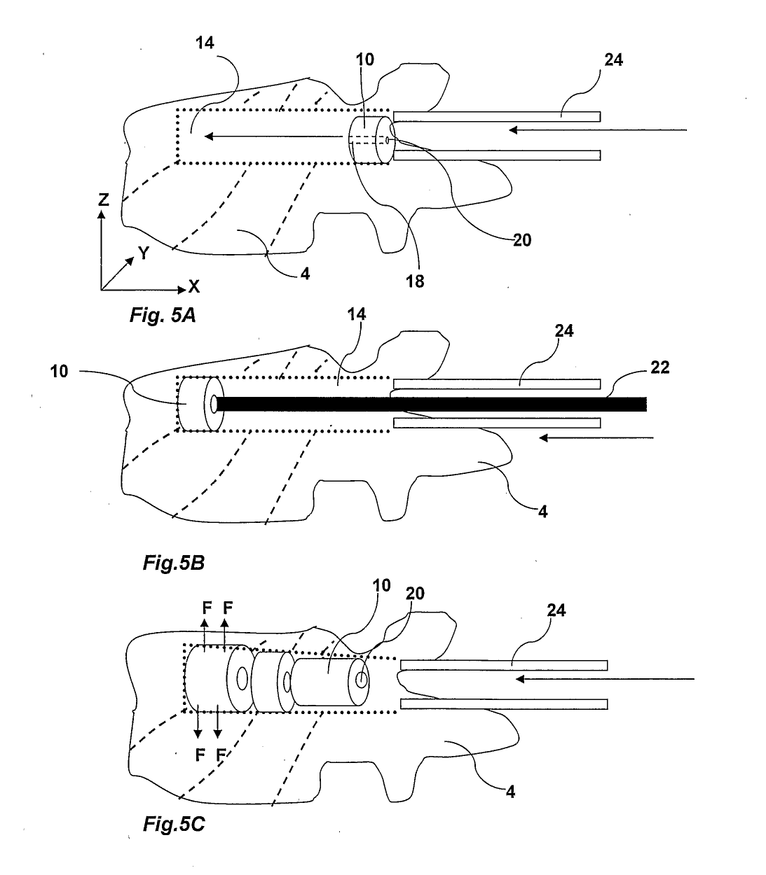

FIGS. 5A-5C are cross sectional side views of a collapsed vertebral body illustrating insertion of an embodiment of spine jack including three support elements, each support element being inserted and relaxed before inserting and relaxing the next support element;

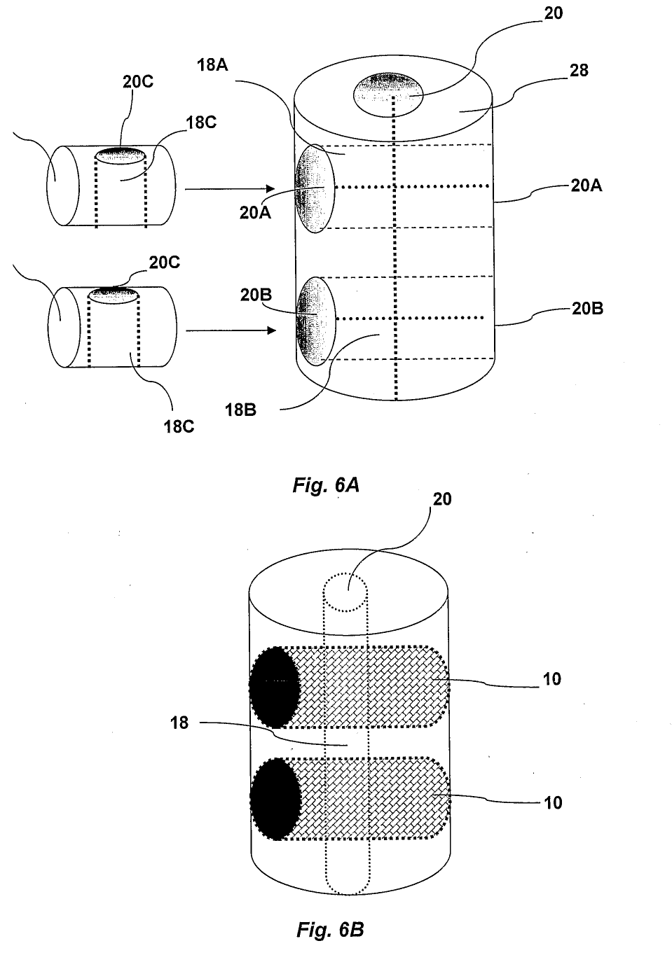

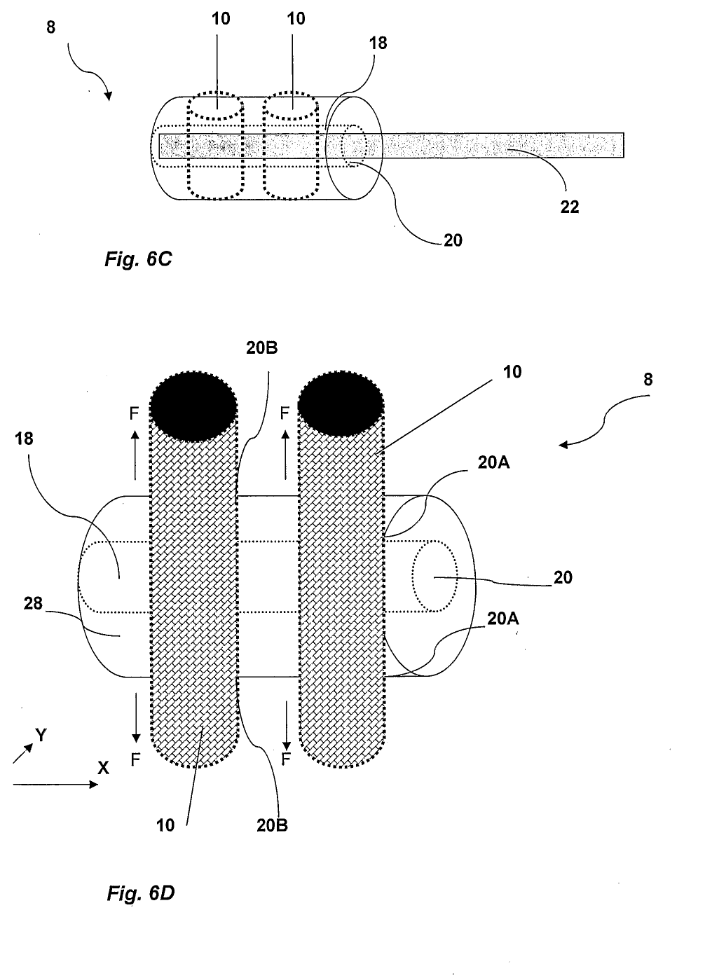

FIG. 6A shows an exploded perspective view and from the side of a further embodiment of spine jack illustrating two orientated cylindrical shaped support elements received within recesses in a side wall of a non-smp holding cylinder, the holding cylinder providing a buffer region absorbing radial forces exerted by the relaxing support elements and directing expansion in a single plane;

FIG. 6B is a similar view of the spine jack of FIG. 6A showing the orientated cylindrical support elements within the holding cylinder;

FIG. 6C is a similar view of the spine jack of FIG. 6B showing a heating probe inserted into the holding cylinder and through each support element delivering heat to relax the support elements;

FIG. 6D is a similar view of the spine jack of FIG. 6C showing relaxed support elements expanded along a single plane;

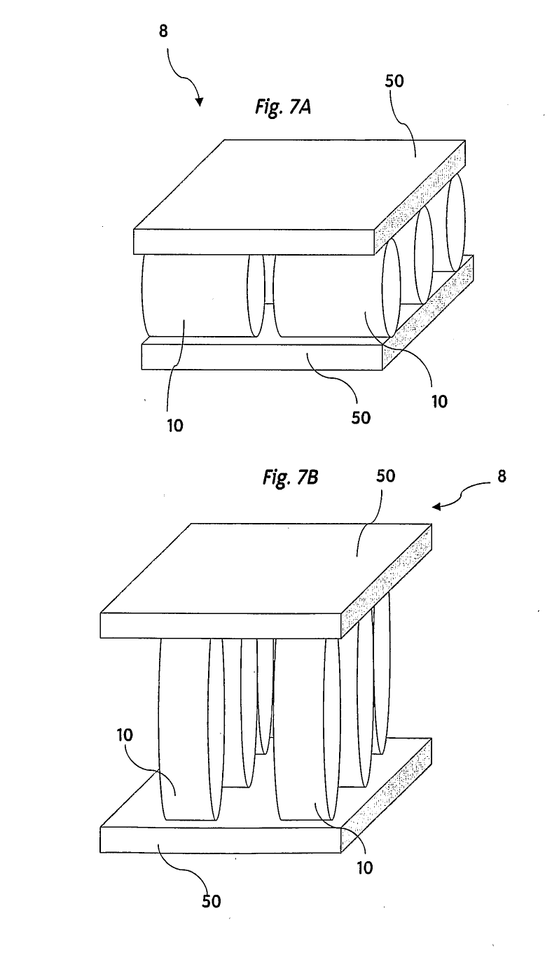

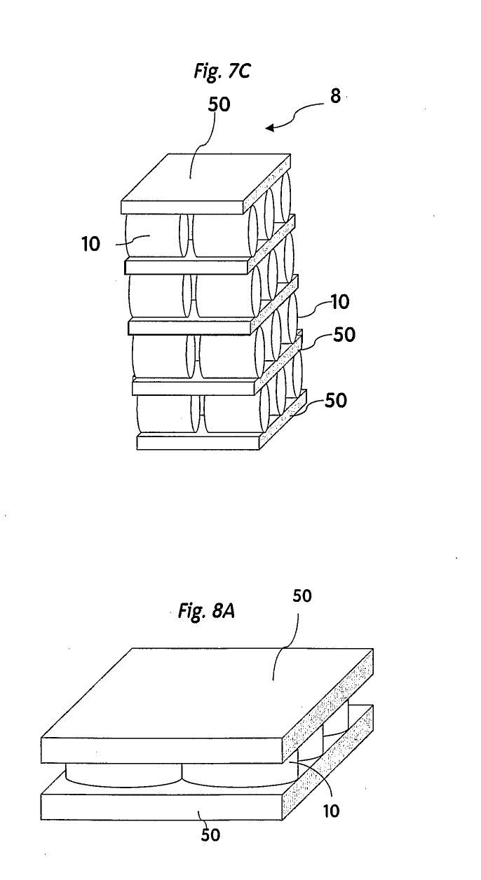

FIG. 7A is a perspective view and from the side of an embodiment of spine jack showing a single layer of two rows of three support elements sandwiched between upper and lower load bearing plates of fixed shape material which interface between the support elements and the load bearing surface of the upper and lower vertebral discs, the support elements shown here in an orientated state;

FIG. 7B is a perspective view and from the side of the spine jack of FIG. 7A showing the support elements in their relaxed form;

FIG. 7C is a perspective view and from the side of an embodiment of spine jack similar to that of FIG. 7A comprising multiple layers;

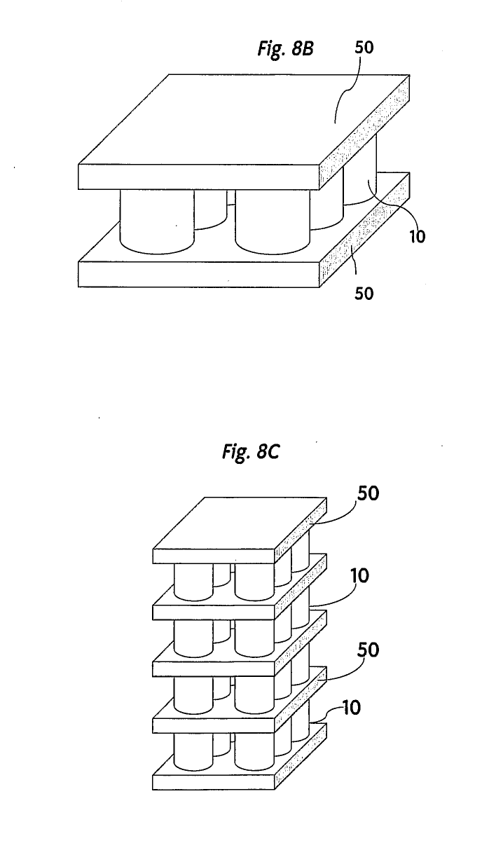

FIGS. 8A-8C are perspective views and from the side similar to those of FIGS. 7A to 7C of an alternative embodiment of spine jack showing supporting elements of a different size and orientation; and

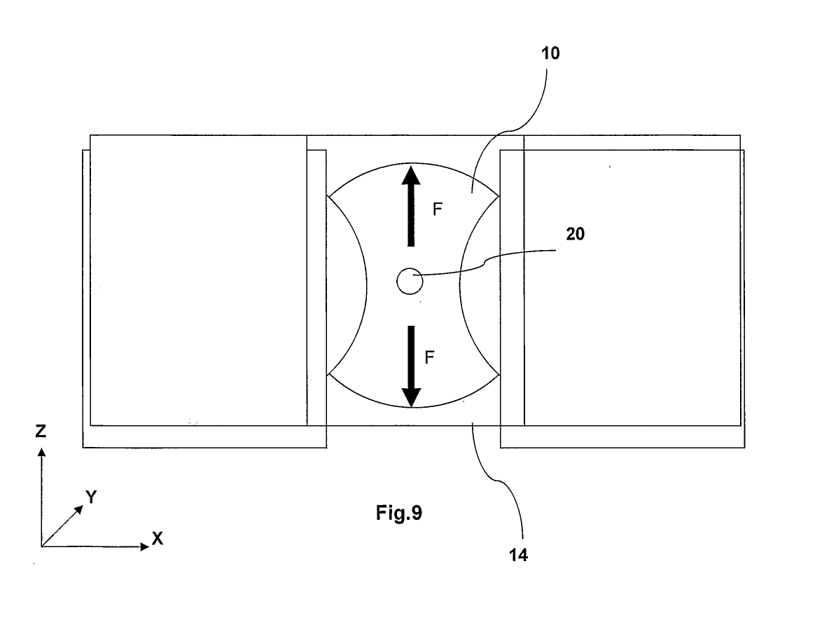

FIG. 9 shows a cross sectional side view of an embodiment of spine jack located within a cavity of a fractured vertebral body, the side walls of the support element being concave in nature.

Referring to the drawings and initially to FIGS. 1A to 1D, there is illustrated a portion of a spinal column generally indicated by the reference numeral 1. The spinal column 1 shows a number intervertebral discs 2 spaced by vertebral bodies 4. As shown in FIG. 1A, one of the vertebral bodies 4 has a fracture 6 which has lead to the collapse of the vertebral body 4. FIG. 1B is a magnified view of the collapsed vertebral body 4 having a spine jack, indicated generally by the reference numeral 8, inserted into the fracture 6. In this embodiment, the spine jack 8 includes two cylindrical support elements 10 of shape memory polymer which have been orientated along their longitudinal axis or X-axis as illustrated in the drawings. Once the spine jack 8 has been inserted into the fracture 6, the support elements 10 are relaxed by applying heat thereto using a heating probe (not shown). Relaxation of the support elements 10 causes them to expand along the z-axis or plane restoring the height of the vertebral body 4. This is shown most clearly in FIGS. 1C and 1D.

FIGS. 1A to 1D provide a general overview only of the spine jack 8 of the present invention and how it works. Further detail will be provided when, describing more detailed embodiments below. By way of explanation and to provide clarity, the axes or planes referred to throughout the description include the X, Y and Z planes. The Z-plane refers to that plane whose direction is generally parallel to the longitudinal axis of the spinal column. The X, Y and Z planes are mutually perpendicular and are employed throughout to aid description of 3D spatial arrangements of portions of the spinal column and the spine jack 8 of the present invention and in particular to describe the direction of the force of the relaxing spine jack 8 with respect to the spinal column and portions thereof. Reoccurring features throughout the description will be described using the same reference numerals.

Referring now to FIGS. 2A to 2E there is shown an embodiment of the present invention illustrating more detail. The spine jack 8 of this embodiment is generally in the shape of a cylindrical rod comprising two component parts including the support element 10 and a delivery aid 16. The support element 10 comprises SMP which has been orientated by stretching along its longitudinal axis. The cylindrical rod shaped spine jack 8 has a passageway 18 running centrally along the length of the spine jack 8 terminating in an opening 20, at a free end of the delivery aid 16, for receiving a heating probe 22 powered from an external power unit 23. The fractured site 6 of the collapsed vertebral body 4, having compression fractures 12 indicated by broken lines, is prepared by drilling a cavity 14 within the vertebral body 4 for receiving the spine jack 8. The prepared cavity 14 includes a cylindrical cavity access passageway 15 through the outer cortical bone.

Prior to use, the diameter of the spine jack 8 is generally equal to the diameter of the cylindrical cavity access passageway 15 and the support element 10 of the spine jack 8 is orientated. In use, the support element 10 of the spine jack 8 is inserted into the cylindrical cavity access passageway 15 using an appropriately sized trocar 24 and plunger 26. The plunger 26 pushes the spine jack 8 from the trocar and into the cavity 14 such that the support element 10 is fully inserted into the cavity 14 and the delivery aid 16 resides snugly within the cylindrical cavity access passageway 15. The passageway 15 holds the delivery aid 16 in place, effectively suspending and supporting the orientated support element 10 in place within the cavity 14.

Once the spine jack 8 is located in place, the trocar 24 guides the heating probe 22 through the opening 20 of the spine jack 8 and along the spine jack passageway 18. The support element 10 which comprises orientated shape memory polymer relaxes when heat is transferred from the heating probe 22 to the support element 10. When the supporting element 10 relaxes, it expands along the Z-plane, i.e. along the longitudinal axis of the spine. The force of the expanding support element 10, indicated by the arrows labeled F, raises the height of the collapsed vertebral body 4. This is shown most clearly in FIG. 2D. FIG. 2E clearly illustrates the spine jack 8 after heating wherein the support element 10 is fully relaxed.

It will be appreciated that whilst most of the expanding force of the support element 10 will occur along the Z-plane, some expanding force may occur along the X and Y planes. The expanding force in the X and Y planes is absorbed by a buffer region, which in this embodiment is empty space and is indicated generally by the reference numeral 28. This is shown most clearly in FIG. 2F and FIG. 2G.

Referring now to FIGS. 3A to 3C there is shown a further embodiment of the present invention. The spine jack 8 of this embodiment and method of insertion into the prepared cavity 14 are identical with the embodiment described above and illustrated in FIG. 2A to FIG. 2G with some modification to the support element 10 and method of relaxing the support element 10 which will now be described. The supporting element 10 is modified to include open ended through channels 30 extending perpendicular to the longitudinal axis of the support element 10. When the spine jack 8 is inserted into the cavity 14, the through channels 30 are aligned in parallel with the Z-Plane. Exothermic bone cement 32 is delivered with a syringe 34 through the opening 20 of the spine jack 8 where it moves along the passageway 18 to the support element 10. As the cement 32 cures, energy is released in the form of heat. This heat stimulates relaxation and thus expansion of the support element 10 in a similar fashion to the embodiment described above and illustrated in FIG. 2A to FIG. 2G. During expansion of the support element 10, the diameter of the channels 30 narrow somewhat and the cement 32 is pushed by the force of the contracting channels 30 through openings 36 and 38 located on upper and lower surfaces 40 and 42 respectively of the supporting element 10. In this way, the cement 32 forms a layer on both the upper and lower surfaces 40 and 42 of the support element 10. These layers serve to bond and integrate the expanded support element 10 with the surrounding bone of the vertebral body 4.

It will be appreciated that where further contraction of the channels 30 is required, the channels 30 may be orientated by stretching them in the radial direction. When the support element 10 is relaxed, the channels 30 will contract to a greater degree than they would if they were not orientated.

It will also be appreciated that a sufficient amount of cement 32 could be injected so that the cement will leak over the edges of the upper surface 42 of the supporting element 10 and into the buffer region 28 providing improved bonding, stabilization and integration of the supporting element 10 with the surrounding bone.

It will be further appreciated that the supporting element may include further channels which direct the cement towards the buffer region 28.

It is envisaged that, instead of using bone cement, the support element 10 could be relaxed in a similar manner as with the embodiment described above and illustrated in FIG. 2A to FIG. 2G. After relaxing the support element 10 in this way, the bone cement 32 could then be injected as described above and under sufficient pressure so the cement will travel along the passageway 18 and be pushed through the channels 30 towards the openings 36 and 38 to aid bonding and integration at the interface formed between the upper and lower surface 40, 42 and the surrounding bone. Instead of bone cement, a biological glue or other integration promoting agent could be used, or a mixture thereof.

Referring now to FIG. 4A and 4B there is shown a further embodiment of the spine jack 8 of the present invention. In this embodiment, the outer surfaces of the spine jack 8 are coated with a flexible bonding material 44 indicated by the thick black boarder line. The bonding material can be polycaprolactone (PCL) or polyurethane for example. The bonding material 44 partially melts from the energy used to relax the support element 10 and creates a bonding medium between the support element 10 and the surrounding bone of the cavity 14. In every other respect, the embodiment is similar with the embodiments described above and illustrated in FIG. 2A to FIG. 2G and FIG. 3A to 3C.

FIG. 5A to 5C describes an alternative embodiment of spine jack 8 according to the present invention. In this embodiment, the spine jack 8 comprises a plurality of support elements 10 of cylindridal shape and having a through passageway 18 running centrally along the length of the support element 10, the passageway 18 terminating at both ends of the support element in an opening 20 for receiving a heating probe 22. In use, a support element 10 is inserted into the prepared cavity 14 in a similar fashion as described above. Once the support element 10 is in place within the cavity 14, the heating probe 22 is inserted through the opening 20 and into the passageway 18 where heat is effectively delivered to the orientated support element 10. This process is repeated for each support element 10. In this way, the collapsed vertebral body 4 can be raised in sections and by degrees, providing a further element of control to the direction and degree of force used to repair the collapsed vertebral body 4. Each of the support elements 10 may also be subjected to different degrees or levels of orientation and relaxation imparting even further control over the direction and degree of force used to repair the collapsed vertebral body 4.

FIGS. 6A to 6D illustrate an alternative embodiment of spine jack 8. In this embodiment, the buffer region 28 is a substantially solid, non shape-changing, cylindrical cavity filler. The cylindrical filler 28 has a passageway 18 running centrally along the length thereof and terminates in an opening 20, for receiving a heating probe 22. The filler 28 also has two support element receiving passageways 18A and 18B terminating at both ends in openings 20A and 20B respectively. The longitudinal axes of the passageways 18A and 18B are generally at right angles to the longitudinal axis of the passageway 18. Each of the cylindrical support elements 10 include a further passageway 18C which extends across the support element 10 at a right angle to the longitudinal axis thereof. The passageway 18C terminates at both ends in an opening 20C to allow passage of the heating probe 22 therethrough. The outer diameter of the support element 10 is generally equal to the inner diameter of the passageway 20A and 20B. This is shown most clearly in FIG. 6A.

In use, the support elements 10 are located and fit snugly within the passageways 20A and 20B of the filler 28. The support elements 10 are positioned within the passageways 20A and 20B such that the passageway 18C of each support element 10 is aligned with the passageway 18 of the filler 28. This allows for the passage of the heating probe 22 along the entire passageway 18. This is shown most clearly in FIG. 6C. The heating probe 22 transfers energy to the orientated support elements 10 which on relaxing expand through the openings 20A and 20B along the Z-plane. The walls of the passageways 18A and 18B fit snugly around the support elements 10 preventing expansion along the X and Y planes. The snug fit of the walls of the passageways 18A and 18B direct the expansion which may otherwise occur in the X and Y planes along the Z plane, thus further improving expansion along the Z-plane and thus the restoring force applied to the collapsed vertebral body 4 improving the inherent repairing capability of the spine jack 8 to restore the vertebral body 4 to its original height.

An alternative embodiment of spine jack 8 is shown in FIGS. 7A to 7C. The spine jack 8 includes a single layer of two rows of three cylindrically shaped support elements 10 sandwiched between upper and lower load bearing plates 50 of fixed shape material which in use, interface between the support elements 10 and the load bearing surface of the upper and lower vertebral discs (not shown). The support elements 10 of the spine jack 8 are shown in FIG. 7A in an orientated form and in FIG. 7B in a relaxed or expanded form. FIG. 7C illustrates the spine jack 8 of FIG. 7A having a plurality of layers. In use, the relaxed support elements 10 of the spine jack 8 transfer their expansion force to the load bearing plates 50 which in turn transfer the expansion force to the vertebral body 4 to restore its height. In this way, the force from the expanding support elements 10 is transferred more evenly over the vertebral discs 4.

FIGS. 8A to 8C illustrate an embodiment of spine jack 8 wherein the support elements 10 have a different size and configuration.

FIG. 9 shows a cross sectional side view of a spine jack 8 located within a cavity 14. The support element 10 is cylindrical in shape having concaved side walls or areas of reduced thickness in both the X and Y planes. When the support element 10 is relaxed, an expansion force is transferred to the vertebral body 4 along the Z plane only. This is due to the particular shape of the support element.

It will be appreciated that various modifications of spine jack are possible within the scope of the present invention. For example, the support elements can be of any suitable shape and size. The load bearing plates may also comprise shape memory polymer which may be orientated. The support element can be cannulated to form a passageway to receive a heating probe as described above or the support element can be formed with an internal heat conducting element which can be connected, by heat conducting wires for example, to an external power unit. The internal heat conducting element can remain in-situ or be removed following relaxation of the support element. Alternatively the support element can be formed of a shape memory polymer whose Tg is body temperature (37° C.). In this way, the temperature of the body fluid would relax the support element and no further input of energy would be required.

It will also be appreciated that the device of the present invention can be used to repair other tissue fractures other than of the spine.

It's envisaged that the spine jack may include one or more active agents. Suitable active agents include growth factors, bone morphogenic proteins, antibiotics, anti-inflammatories, angiogenic factors, osteogenic factors, monobutyrin, omental extracts, thrombin, modified proteins, platelet rich plasma/solution, platelet poor plasma/solution, bone marrow aspirate, and any cells sourced from flora or fauna, such as living cells, preserved cells, dormant cells, and dead cells. It will be appreciated that other bioactive agents known to one of ordinary skill in, the art may also be used. The active agent can be incorporated into the polymeric shape memory material, to be released during the relaxation or degradation of the polymer material. Advantageously, the incorporation of an active agent can act to combat infection at the site of implantation and/or to promote new tissue growth.

The invention is not limited to the embodiments and modifications hereinbefore described which may be varied in both construction and detail within the scope of the appended claims.

1. A spine jack, insertable into a bone cavity for treating an injured or collapsed vertebral body of a spinal column, comprising at least one support element having shape memory properties, the spine jack being adapted such that the support element exerts a force to the vertebral body along a single support plane.

2. The spine jack of claim 1, wherein the support element comprises a shape memory polymer.

3. The spine jack of claim 2, wherein the shape memory polymer comprises an orientated shape memory polymer.

4. The spine jack of claim 1, wherein the spine jack further comprises a buffer region surrounding at least a portion of the support element for absorbing forces exerted by the support element in planes other than said single support plane.

5. The spine jack of claim 4, wherein the buffer region comprises a medium having a density of between 1.168 kg/m³ and 50 kg/m³ measured at 25° C. and 100 kPa.

6. The spine jack of claim 5, wherein the buffer region comprises a compressible matrix.

7. The spine jack of claim 4, wherein the buffer region comprises a non shape changing material in the form of a plug shaped to fit snugly within the bone cavity.

8. The spine jack of claim 7, wherein the plug comprises at least one recess for receiving the at least one support element.

9. The spine jack of claim 1, wherein the at least one support element is sandwiched between two load bearing interfaces.

10. The spine jack of claim 1, wherein the at least one support element comprises a passageway for receiving an energy transfer probe.

11. An apparatus for treating an injured or collapsed vertebral body of a spinal column, comprising a spine jack of claim 1, means for delivering the spine jack to the vertebral body and means for stimulating the shape memory properties of the support element.

12. The apparatus of claim 11, wherein the means for delivering the spine jack comprises a cannula or trocar.

13. The apparatus of claim 11, wherein the stimulating means comprises a heating probe.

14. A method of repairing a fractured vertebral body comprising:
introducing the spine jack of claim 1 into the fractured vertebral body; and
stimulating molecular motion of the at least one support element.

15. The method of claim 14, further comprising preparing the fractured vertebral body by forming a cavity therein.
